(19) United States
(12) Patent Application Publication
(10) Pub. No.: US 2009/0112069 A1
(43) Pub. Date: Apr. 30, 2009

(54) TREND PREDICTION DEVICE

(75) Inventors: Nobuhiro KANAMORI, Moriguchi City (JP); Yoshihisa Fujiwara, Moriguchi City (JP)

Correspondence Address: MOTS LAW, PLLC, 1629 K STREET N.W., SUITE 602, WASHINGTON, DC, 20006-1635, US

(73) Assignee: SANYO ELECTRIC CO., LTD., Moriguchi City (JP)

(21) Appl. No.: 12/237,760

(22) Filed: Sep. 25, 2008

(30) Foreign Application Priority Data
Oct. 31, 2007 (JP) JP2007-284542

(51) Int. Cl. A61B 5/00

(52) U.S. Cl. 600/300

(57) ABSTRACT

A trend prediction device that is versatile and capable of improving the accuracy of predicting a trend in a user's physical condition is provided. The trend prediction device includes: a sensor-data converter configured to convert sensor data detected by a sleep sensor into a sleep-related parameter for making a physical-data-trend judgment; a parameter acquisition unit configured to acquire a lifestyle-related parameter that indicates an action of the user during a non-sleeping period, and possibly changing the physical-data trend; and a parameter comparator configured to compare the sleep-related and the lifestyle-related parameters with respective reference parameters. The trend prediction device is configured to judge whether the physical data has an increase or a decrease in trend on the basis of the comparison result of the sleep-related and the lifestyle-related parameters with their respective reference parameters.

### CROSS REFERENCE TO RELATED APPLICATIONS

This application claims priority based on 35 USC 119 from prior Japanese Patent Application No. P2007-284542 filed on Oct. 31, 2007, the entire contents of which are incorporated herein by reference.

### BACKGROUND OF THE INVENTION

1. Field of the Invention

The present invention relates to a trend prediction device configured to predict a variation trend of physical data showing the physical state of a user.

2. Description of Related Art

In recent years, aging of the population has been advancing and the disease structure has been changed in our country, so that the importance of promoting health has grown. What is required accordingly is the development of appropriate conditions for actively promoting the health and preventing diseases. Under these circumstances, attention of the public has been attracted to a technique of detecting and managing biological data of a user during sleeping (for example, breathing, heart rate, body movement, and the like).

Japanese Patent No. 3453877 (Patent Document 1) discloses a technique of detecting the biological data of a user during sleeping. In the technique, vibration frequencies are obtained by a vibration sensor disposed in the bed, and then are analyzed by Fourier conversion to detect the biological data of the user.

In addition, in the method disclosed in Patent Document 1, the resonant frequency of the vibration model composed of the bed and the human body is obtained from the analysis of the vibration frequencies. In addition, the body weight of the user is calculated by the spring constant for the bed. Then, from the history of the calculated body weight, a judgment is performed as to whether the body weight of the user is increasing or decreasing.

The method of Patent Document 1, however, requires actual measurement of the body weight to judge whether the body weight of the user has increased or decreased. For this reason, the body-weight-trend prediction method disclosed in Patent Document 1 can be effectively used only with a bed that has a known spring constant. Thus, there is a problem that the method is not versatile.

Incidentally, the trend of the user's physical conditions (for example, blood pressure, blood-sugar level, body weight, and body-fat percentage) may possibly be changed by the action (for example, eating and exercising) of the user during a non-sleeping period. The method of Patent Document 1, however, predicts the trend of the user's physical condition by means only of the biological data detected while the user is sleeping, so the prediction has a low accuracy.

### SUMMARY OF THE INVENTION

An aspect of the invention provides a trend prediction device that indicates the physical conditions of a user, which comprises: a first parameter acquisition unit configured to acquire values of a first judgment parameter for the user's sleep, which is a quantified factor affecting the physical data; a second parameter acquisition unit configured to acquire values of a second judgment parameter for an action of the user during non-sleeping, which is a quantified factor affecting the physical data; a parameter accumulator configured to accumulate the acquired values of the first and second judgment parameters; a reference parameter calculator configured to calculate a first reference parameter from the values of the first judgment parameter accumulated in the parameter accumulator and to calculate a second reference parameter on the basis of the values of the second judgment parameter accumulated in the parameter accumulator; a parameter comparator configured to compare each of the values of the first judgment parameter with the first reference parameter and to compare each of the values of the second judgment parameter with the second reference parameter; and a trend judgment unit configured to detect an increased or decreased trend in a parameter by comparing each value of the first judgment parameter with the first reference parameter and from comparing each value of the second judgment parameter with the second reference parameter.

According to the trend prediction device, the trend of the physical data showing the body condition of the user is judged not only by the first judgment parameter, which is information on the sleep of the user and which is obtained by quantifying the factor affecting the physical data but also by the second judgment parameter, which is related to the user's actions while he or she is not sleeping and which is obtained by quantifying the factor affecting the physical data.

Accordingly, the trend of the physical data can be judged by taking account of the user's conditions both while he or she is sleeping and while he or she is not sleeping. As a consequence, the accuracy of predicting the variation trend of the physical data can be improved.

In addition, whether the physical data has an increase or a decrease in trend is judged on the basis of the result of the comparison between the first judgment parameter and the first reference parameter as well as on the basis of the result of the comparison between the second judgment parameter and the second reference parameter. Further, no information on the bed is necessary. For these reasons, the trend prediction device is highly versatile.

In the trend prediction device, the first judgment parameter is preferably a parameter obtained by converting sensor data which are data detected by a sleep sensor disposed in a bed.

According to such a trend prediction device, the first judgment parameter is obtained by converting the sensor data. For this reason, the accuracy of the first judgment parameter can be enhanced. As a consequence, the prediction accuracy can be enhanced.

The trend prediction device preferably further comprises a judgment-result notification unit configured to notify the user of the result of a judgment performed by the trend judgment unit.

According to such a trend prediction device, the user can promote health and prevent disease by notifying the user whether the physical data have an increased trend or a decreased trend.

In the trend prediction device, the parameter comparator preferably calculates a set of ratios of the first reference parameter with the first judgment parameter values and a set of ratios of the second reference parameter with the second judgment parameter values. In addition, the trend judgment unit preferably comprises: a mean-value calculator configured to calculate a mean value of each set of ratios calculated by the parameter comparator; and a prediction unit configured to determine a degree of increased or decreased trend of physical data from the mean values calculated by the mean-value calculator.

According to such a trend prediction device, what is judged is not only whether the physical data has an increased trend or a decreased trend but also the degree of the increase in trend of the physical data or the degree of the decrease in trend of the physical data. For this reason, the accuracy of predicting the variation trend of the physical data can be improved.

The trend prediction device preferably further comprises a parameter accumulator configured to accumulate both the first judgment parameter acquired by the first parameter acquisition unit and the second judgment parameter acquired by the second parameter acquisition unit; and a reference parameter calculator configured to calculate, as the first reference parameter, a representative value of the past first judgment parameter accumulated in the parameter accumulator and to calculate, as the second reference parameter, a representative value of the past second judgment parameter accumulated in the parameter accumulator. In addition, the representative value is preferably any one of the mean value, the median value, and the mode value.

According to such a trend prediction device, the reference parameter can be adapted to the user by calculating the reference parameter from the past parameter. For this reason, the trend prediction device can be more versatile. In addition, the accuracy of predicting the variation trend of the physical data can be improved by use of the reference parameter that is adapted to the user.

The trend prediction device preferably further comprises a parameter extractor configured to extract, depending on a kind of the physical data to be predicted, at least one of a plurality of values of the first judgment parameters obtained by the first parameter acquisition unit and at least one of a plurality of the values of second judgment parameters obtained by the second parameter acquisition unit. In addition, the parameter comparator preferably compares the first judgment parameter extracted by the parameter extractor with the first reference parameter and compares the second judgment parameter extracted by the parameter extractor with the second reference parameter.

According to such a trend prediction device, the parameter to be used is extracted depending on the kind of the physical data to be predicted. For this reason, the parameter that is suitable for each of the physical data can be used. As a consequence, the accuracy of predicting the variation trend of the physical data can be improved.

The trend prediction device preferably further comprises: a table holding unit configured to hold a parameter-to-be-used table that associates physical data to be predicted with the first judgment parameter and the second judgment parameter that are to be extracted by the parameter extractor; a factor detector configured to detect, among the first judgment parameters and the second judgment parameters, a factor parameter that causes a change in the trend of the physical data, from a judgment by the trend judgment unit; and a table updater configured to update the parameter-to-be-used table by use of the factor parameter detected by the factor detector.

According to such a trend prediction device, the parameter-to-be-used table can be optimized by detecting the factor parameter that is a factor causing a change in the trend of the physical data and then by updating the parameter-to-be-used table. For this reason, the trend prediction device can be more versatile. In addition, the accuracy of predicting the trend of the physical data can be improved.

The trend prediction device preferably further comprises a factor notification unit configured to notify the user of the factor parameter detected by the factor detector.

According to such a trend prediction device, the user is notified of the factor parameter that is a factor causing a change in the trend of the physical data. For this reason, the user can promote the health and prevent the disease actively.

The trend prediction system is highly versatile, and is capable of improving the accuracy of predicting the variation trend of the body conditions of the user.

### BRIEF DESCRIPTION OF THE DRAWINGS

FIG. 1 is a diagram illustrating the general overall configuration of a trend prediction system according to a first embodiment.

FIG. 2 is a diagram illustrating the functional-block configuration of a trend prediction device according to the first embodiment.

FIG. 3 is a table configuration diagram showing an example of a parameter-to-be-used table according to the first embodiment.

FIG. 4 is a diagram for explaining the function of a parameter comparator according to the first embodiment.

FIG. 5 is a table configuration diagram showing an example of a table for judgment according to the first embodiment.

FIG. 6 is a flowchart illustrating the general operation of the trend prediction device according to the first embodiment.

FIG. 7 is a flowchart illustrating a series of processing performed by a sensor-data converter in order to determine whether the user starts to sleep or not according to the first embodiment.

FIG. 8 is a flowchart illustrating a series of processing performed by the sensor-data converter in order to determine the depth of the user's sleep according to the first embodiment.

FIG. 9 is a flowchart illustrating a series of processing performed by the sensor-data converter in order to determine the length of the apneic period according to the first embodiment.

FIG. 10 is a flowchart illustrating a series of processing performed by the sensor-data converter in order to determine whether the user is awake or waking up the according to the first embodiment.

FIG. 11 is a waveform diagram (part 1) for describing the parameter conversion processing performed by the sensor-data converter according to the first embodiment.

FIG. 12 is a waveform diagram (part 2) for describing the parameter conversion processing performed by the sensor-data converter according to the first embodiment.

FIG. 13 is a flowchart illustrating the procedure of a series of trend prediction processing performed by the trend prediction device according to the first embodiment.

FIG. 14 is a diagram illustrating the functional-block configuration of a trend prediction device according to a second embodiment.

FIG. 15 is a table configuration diagram showing an example of information stored in a comparative-result storage according to the second embodiment.

FIG. 16 is a flowchart illustrating the procedure of a series of factor analysis processings performed by the trend prediction device according to the second embodiment.

FIGS. 17A and 17B are table configuration diagrams showing examples of a reference-parameter table according to other embodiments.

### DETAILED DESCRIPTION OF EMBODIMENTS

The first and second embodiments will be described below with reference to the accompanying drawings. In the following description, parts or units that are identical or similar to each other across the first and the second embodiments will be given identical or similar reference numerals.

### First Embodiment

In the description of the first embodiment, those items given below will be described in the following order: (1) the general overall configuration of a trend prediction system; (2) the configuration of a trend prediction device; (3) the operation of the trend prediction device; and (4) effects.

### (1) General Overall Configuration of Trend Prediction System

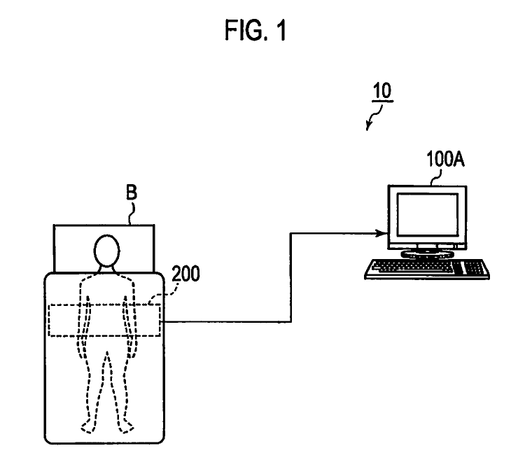

FIG. 1 is a diagram illustrating the general overall configuration of trend prediction system 10 according to this embodiment. As FIG. 1 shows, the trend prediction system 10 includes sleep sensor 200 and trend prediction device 100A that is connected to sleep sensor 200.

Sleep sensor 200 is a thin, non-restrictive sensor, and is installed in bed B. Sleep sensor 200 is configured to detect pressure (vibration and the like) to be applied on sleep sensor 200, and to transmit electric signals (hereafter, referred to as “sensor data”) that show the detection results to trend prediction device 100A.

Trend prediction device 100A may be an information-processing device that includes a CPU, a memory, and the like, and is configured to predict the variation trend (an increase or a decrease in trend) of physical data that shows the physical state of the user. In this embodiment, the physical data includes parameters such as the blood pressure, the blood-sugar level, the body weight, and the body-fat percentage, the parameters being factors from which the health condition of the user can be judged.

Trend prediction device 100A is configured to convert sensor data received from sleep sensor 200 into sleep-related parameters P1, and to predict the trend of the physical data on the basis of sleep-related parameters P1. In this embodiment, sleep-related parameters P1 include the into-bed time, the wake-up time, the sleep-onset time, the final-awakening time, the sleep latency, the total sleep time, the sleep depth, and the sleep apnea syndrome (SAS).

The into-bed time mentioned above is the clock time when the user gets into bed B. The wake-up time is the clock time when the user gets out of bed B. The sleep-onset time is the clock time when the user that has got into bed B actually starts sleeping. The final-awakening time is the clock time when the user finishes sleeping. The sleep latency is the duration of time from the into-bed time to the sleep-onset time. The total sleep time is the duration of time from the sleep-onset time to the final-awakening time. The sleep depth is the depth of sleep of the user.

For the purpose of predicting the variation trend of the physical data, trend prediction device 100A employs, in addition to sleep-related parameter P1, lifestyle-related parameters P2 obtained by quantifying the factors which are related to the action taken by the user while he/she is awake and which can change the trend of the physical data. In this embodiment, lifestyle-related parameters P2 may include calorie intake, fat intake, salt intake, sugar intake, alcohol intake, the amount of smoking, and the amount of exercise.

### (2) Configuration of Trend Prediction Device

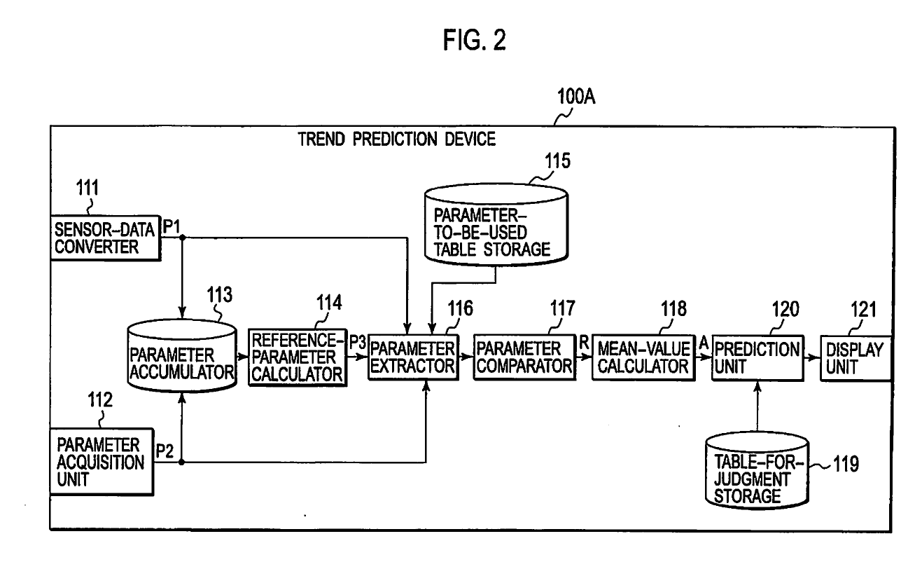

Next, the configuration of trend prediction device 100A will be described with reference to FIGS. 2 to 5.

### (2.1) Functional-Block Configuration of Trend Prediction Device

FIG. 2 is a diagram illustrating the functional-block configuration of trend prediction device 100A. The following description focuses mainly on the points that relate to this embodiment.

As FIG. 2 shows, trend prediction device 100A includes sensor-data converter 111, parameter acquisition unit 112, parameter accumulator 113, reference-parameter calculator 114, parameter-to-be-used table storage 115, parameter extractor 116, parameter comparator 117, mean-value calculator 118, table-for-judgment storage 119, prediction unit 120, and display unit 121.

Sensor-data converter 111 is configured to convert the sensor data into sleep-related parameters P1. Specifically, sensor-data converter 111 is configured to judge the user's body-movement state and respiratory state, and to create sleep-related parameters P1 on the basis of the judgment results.

Parameter acquisition unit 112 is configured to acquire lifestyle-related parameters P2 obtained by quantifying the factors that are related to the action taken by the user while he/she is awake and which can change the trend of the physical data. In this embodiment, parameter acquisition unit 112 may acquire lifestyle-related parameters P2 from the information received from the user though an input device, such as a keyboard and a mouse.

Parameter accumulator 113 is configured to accumulate sleep-related parameters P1 created by sensor-data converter 111 and lifestyle-related parameters P2 acquired by the parameter acquisition unit 112.

Reference-parameter calculator 114 is configured to calculate, as reference parameters P3, the representative value of past sleep-related parameters P1 accumulated by parameter accumulator 113 and the representative value of past lifestyle-related parameters P2 accumulated by parameter accumulator 113. The representative value mentioned above may be any one of the mean value, the median value, and the mode value.

Parameter extractor 116 is configured to extract, in accordance with the type of physical data to be predicted, at least one of multiple sleep-related parameters P1 created by sensor-data converter 111 and at least one of multiple lifestyle-related parameters P2 acquired by parameter acquisition unit 112.

Parameter-to-be-used table storage 115 stores parameter-to-be-used table T1 that associates the kinds of physical data to be predicted with sleep-related parameters P1 and lifestyle-related parameters P2, which are to be extracted by parameter extractor 116.

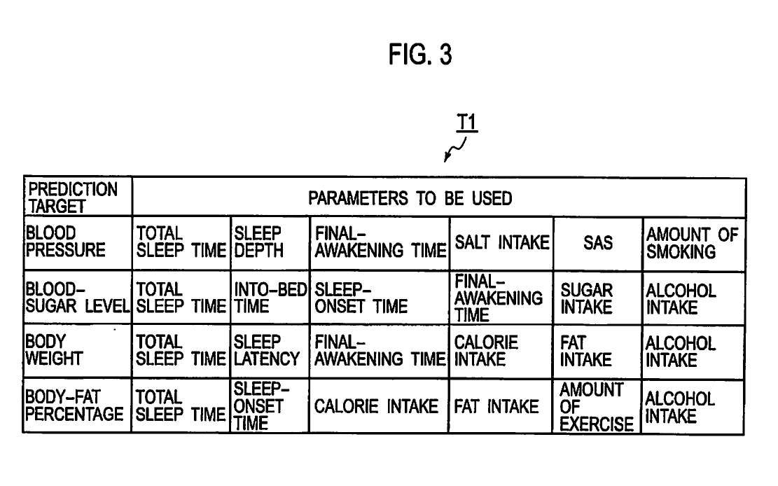

FIG. 3 is a table configuration diagram showing an example of parameter-to-be-used table T1. As FIG. 3 shows, parameter-to-be-used table T1 associates the types of physical data to be predicted with the types of parameters to be used in predicting the trend of the physical data.

For example, the parameters that are used when the trend of blood pressure is predicted are: among sleep-related parameters P1, the total sleep time, the sleep depth, the final-awakening time, and the SAS; and among lifestyle-related parameters P2, the salt intake and the amount of smoking. What should be noted here is that FIG. 3 shows the initial state of parameter-to-be-used table T1, and that parameter-to-be-used table T1 can be updated as will be described in the second embodiment.

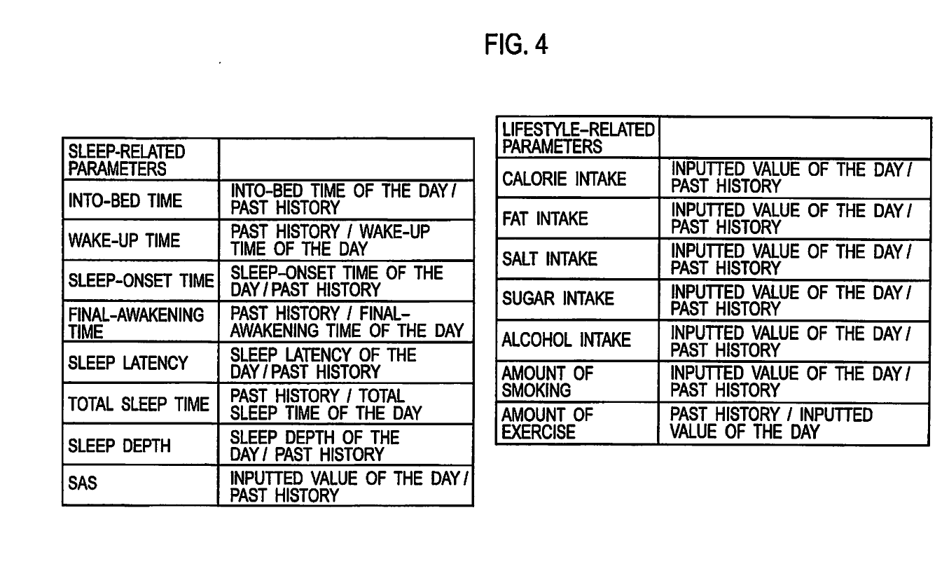

Parameter comparator 117 is configured to compare both sleep-related parameters P1 and lifestyle-related parameters P2, which are extracted by parameter extractor 116, with reference parameters P3. Specifically, as FIG. 4 shows, parameter comparator 117 calculates ratios R of new sleep-related parameters P1 to the representative values of their respective past sleep-related parameters P1, and ratios R of new lifestyle-related parameters P2 to the representative values of their respective past lifestyle-related parameters P2.

Consider an example of trend prediction of the body weight. In this case, ratio R concerning the sleep latency, which is a sleep-related parameter P1 is calculated. For example, the sleep latency of the day is 1.1 times as long as the representative value of the past sleep latency. In addition, ratio R concerning the calorie intake that is one of lifestyle-related parameters P2 is calculated. For example, the calorie intake of the day is 0.9 times as much as the representative value of the past calorie intake.

Mean-value calculator 118 is configured to calculate mean value A of all ratios R that have been calculated by parameter comparator 117. For example, suppose a case where ratio R of the total sleep time is 1.1 times, ratio R of the sleep latency is 1.8 times, ratio R of the final-awakening time is 0.8 times, ratio R of the calorie intake is 1.1 times, ratio R of the fat intake is 1.2 times, and ratio R of the alcohol intake is 1 time. In this case, mean value A is 1.2 times (rounded off to the nearest tenth).

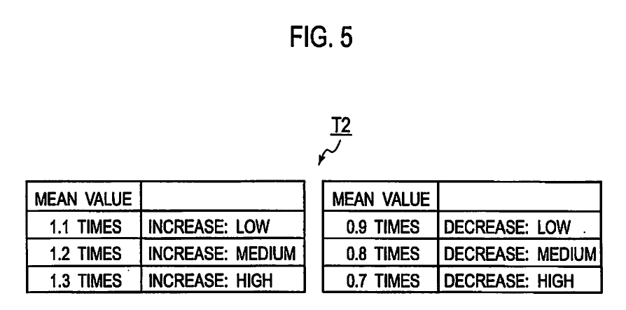

Table for judgment T2, which associates mean value A with the degree of increase or decrease in trend as FIG. 5 shows, is stored beforehand in table-for-judgment storage 119. In the example shown in FIG. 5, the mean value of “1.1 times” is associated with the increase in trend (“low” degree of increased trend), the mean value of “1.2 times” is associated with the increase in trend (“medium” degree of increased trend), and the mean value of “1.3 times” is associated with the increase in trend (“high” degree of increased trend). In addition, the mean value of “0.9 times” is associated with the decreased trend (“low” degree of decreased trend), the mean value of “0.8 times” is associated with the decreased trend (“medium” degree of decreased trend), and the mean value of “0.7 times” is associated with the decreased trend (“high” degree of decreased trend). The mean value of “1 time” is judged as “no-change.” The mean value of either not less than “1.3 times” or not more than “0.7 times” is judged to be a “high” degree of the corresponding trend.

Prediction unit 120 functions as a degree judgment unit configured to judge, on the basis of the table for judgment T2, both the trend and the degree of the trend that correspond to mean value A calculated by mean-value calculator 118. Display unit 121 is configured to display the result of the prediction performed by prediction unit 120. For example, when mean value A of “1.2 times” is obtained in the prediction about the body-weight trend, display unit 121 displays such a message as “medium-degree increase in body weight.”

Note that, in this embodiment, mean-value calculator 118, table-for-judgment storage 119, and prediction unit 120 each may function as a trend judgment unit configured to judge whether the physical data has an increased or a decreased trend. In addition, in this embodiment, display unit 121 may function as a judgment-result notification unit configured to notify the user of the result of the judgment performed by the trend judgment unit.

### (3) Operation of Trend Prediction Device

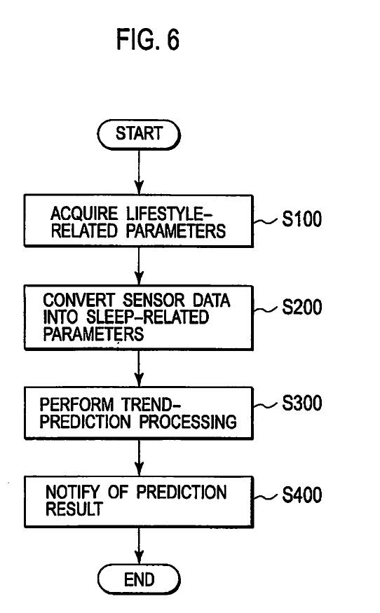

Next, the operation of trend prediction device 100A will be described with reference to FIGS. 6 to 13.

### (3.1) General Operation of Trend Prediction Device

FIG. 6 is a flowchart illustrating the general operation of the trend prediction device 100A.

At step S100, parameter acquisition unit 112 of trend prediction device 100A acquires lifestyle-related parameters P2.

At step S200, sensor-data converter 111 of trend prediction device 100A converts the sensor data acquired by sleep sensor 200 into sleep-related parameters P1. Details of step S200 will be described later. Note that the processing at step S200 may be performed either before step S100 or simultaneously with step S100.

At step S300, trend prediction device 100A executes the processing for predicting the variation trend of physical data. Details of step S300 will be described later.

At step S400, display unit 121 of trend prediction device 100A displays the prediction result obtained at step S300. Note that the display on display unit 121 is not the only way of notification. Instead, notification can be done by use of a speaker (with a sound)

### (3.2) Flow of Parameter Conversion Processing

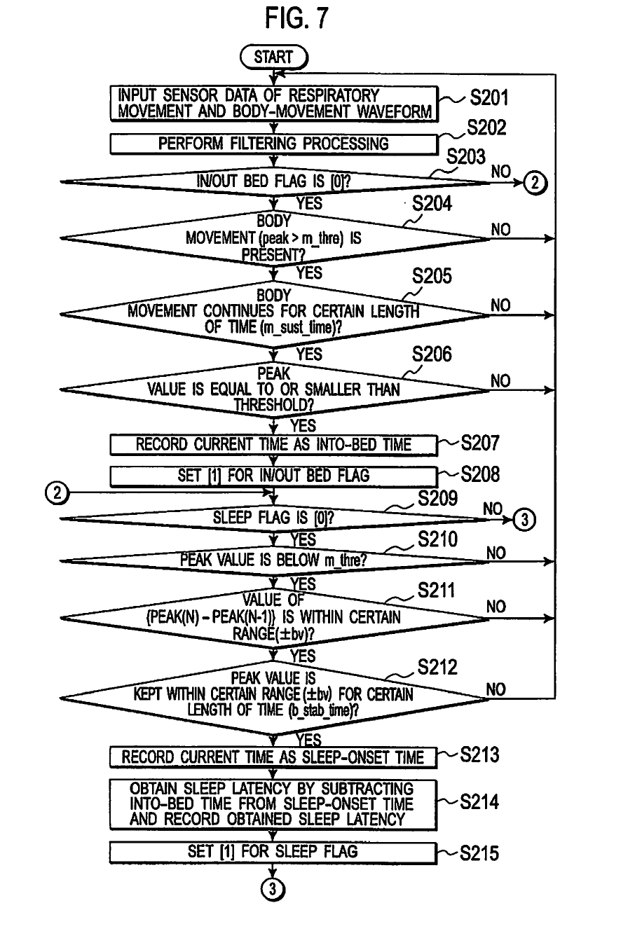

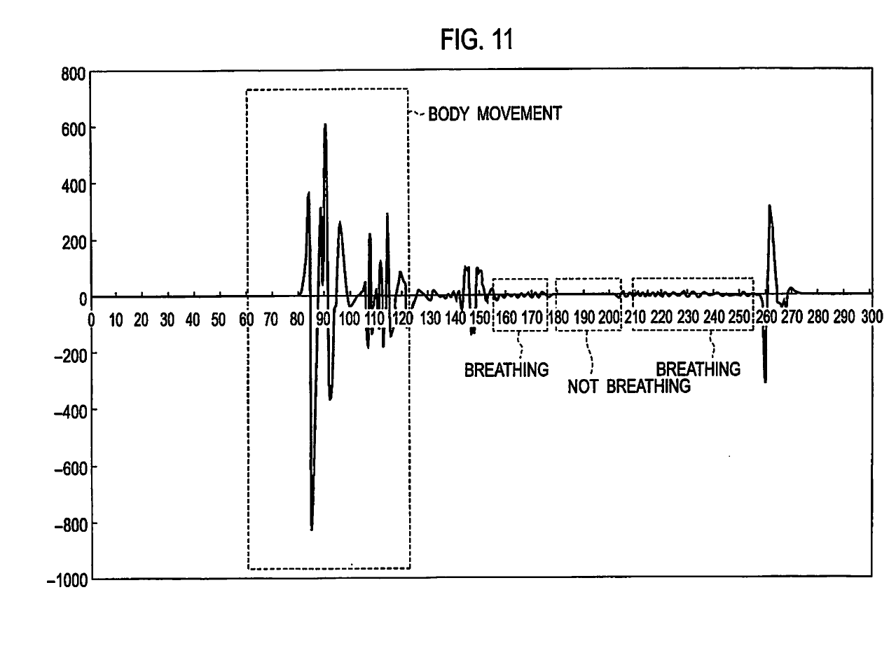

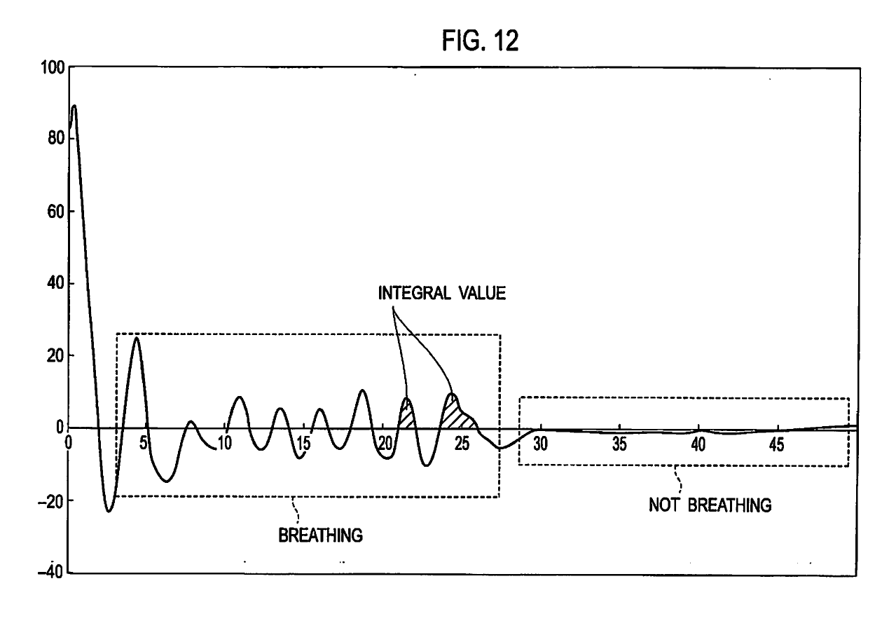

FIGS. 7 to 10 are flowcharts illustrating the procedure of a series of parameter conversion processing executed by sensor-data converter 111. The procedure of a series of parameter conversion processing shown in FIGS. 7 to 10 will be briefly described below with reference to the waveform charts in FIGS. 11 and 12.

At step S201 of FIG. 7, sensor-data converter 111 acquires sensor data on the respiratory-movement and body-movement wave form. At step S202, a noise component is removed, through a filtering process, from the sensor data thus acquired.

At steps S203 to S208, sensor-data converter 111 judges whether the user has got into bed or not. Specifically, the conditions on which sensor-data converter 111 judges that the user has got into bed are: the continuous amplitude representing body movements in the waveform chart shown in FIG. 11 for a certain length of time (m_sust_time); and the subsequent lowering of the peak value of the amplitudes representing body movements down to the threshold value or even lower. Then, the into-bed time, which is one of the sleep-related parameters P1, is recorded. Note that whether or not the amplitude represents body movements can be identified by examining whether or not the peak value of the amplitude (peak) is larger than the threshold value (m_thre).

The processing for judging whether or not the user has got into bed will be described below with reference to the flowchart shown in FIG. 7. Firstly, whether or not the IN/OUT bed flag is zero is judged (at step S203). The IN/OUT bed flag mentioned above is stored in a storage (not illustrated) that is provided in trend prediction device 100A. The IN/OUT bed flag thus stored can be referred to when necessary. Subsequently, sensor-data converter 111 judges whether or not the amplitude representing the body movement is larger than the threshold value (at step S204). The judgment can be made on the basis of whether or not the peak value of the amplitude (peak) representing the body movement is larger than the threshold value (m_thre). When sensor-data converter 111 judges that the peak value is larger than the threshold value, the operational flow proceeds to step S205. Conversely, when sensor-data converter 111 judges that the peak value is smaller than the threshold value, the operational flow proceeds back to step S201. Subsequently, sensor-data converter 111 judges whether or not the amplitude representing the body movement in the waveform chart shown in FIG. 11 continues for a certain length of time (m_sust_time) (at step S205). When such amplitude does not continue for the certain length of time, the operational flow proceeds back to step S201. Conversely, when it is judged that the amplitude representing body movement continues for the certain length of time, it is then judged whether or not the peak value of the amplitude representing the body movement becomes equal to or smaller than the threshold value (at step S206). When the peak value is equal to or larger than the threshold value, the operational flow proceeds back to step S201. Conversely, when the peak value becomes equal to or smaller than the threshold value, it is judged that the user has got into bed. Then, the current clock time is recorded as the into-bed time, which is one of the sleep-related parameters P1 (at step S207). The into-bedtime is stored in parameter accumulator 113. Furthermore, the IN/OUT bed flag is set at 1 (at step S208).

When the IN/OUT bed flag is 1 at step S203, that is, when it is determined that the user is in bed, the sensor-data converter 111 next judges whether or not the user starts sleeping through the processing at steps S209 to S215. Specifically, when the peak value (peak) is smaller than the threshold value (m_thre) in the waveform chart shown in FIG. 11, and at the same time when a state in which the variation of the amplitudes (peak-peak) is small continues for a certain period of time (b_stab_time), it is judged that the user starts sleeping and the sleep-onset time is recorded. In addition, the difference between the into-bed time and the sleep-onset time is recorded as the sleep latency.

The processing for judging whether or not the user has started sleeping will be described below with reference to the flowchart shown in FIG. 7. Firstly, whether or not the sleep flag is 0 is judged (at step S209). The sleep flag is stored in the storage (not illustrated) of trend prediction device 100A, and is referenced when necessary. When it is judged that the sleep flag is not 0, that is, when it has been already judged that the user was asleep, the operational flow proceeds to step S216, which will be described later. Conversely, when the sleep flag is 0, it is then judged whether or not the peak value (peak) of the body-movement waveform obtained at step S201 is equal to or smaller than the threshold value (m_thre) (at step S210). When it is judged that the peak value is larger than the threshold value, the operational flow proceeds back to step S201. Conversely, when the peak value is equal to or smaller than the threshold value, it is then judged whether or not the variation of the peak value of the body-movement waveform (peak (n)-peak (n-1)) is within a predetermined range (bv) (at step S211). When it is judged that the variation is out of the predetermined range (bv), the operational flow proceeds back to step S201. Conversely, when the variation is within the predetermined range (bv), it is then judged whether or not the peak value of the body movements (peak) within the predetermined range (bv) continues for a certain length of time (b_stab_time) (at step S212). When it is judged that such a peak value does not continue for the certain length of time (b_stab_time), the operational flow proceeds back to step S201. Conversely, when it is judged that such a peak value continues for the certain length of time (b_stab_time), it is judged that the user starts sleeping, and the current clock time is recorded as the sleep-onset time (at step S213). The data on the sleep-onset time is stored in parameter accumulator 113. Subsequently, the sleep latency is obtained by subtracting the into-bed time obtained at step S207 from the sleep-onset time (at step S214). The data on the sleep latency is stored in parameter accumulator 113. Then, the sleep flag is set at 1 (at step S215).

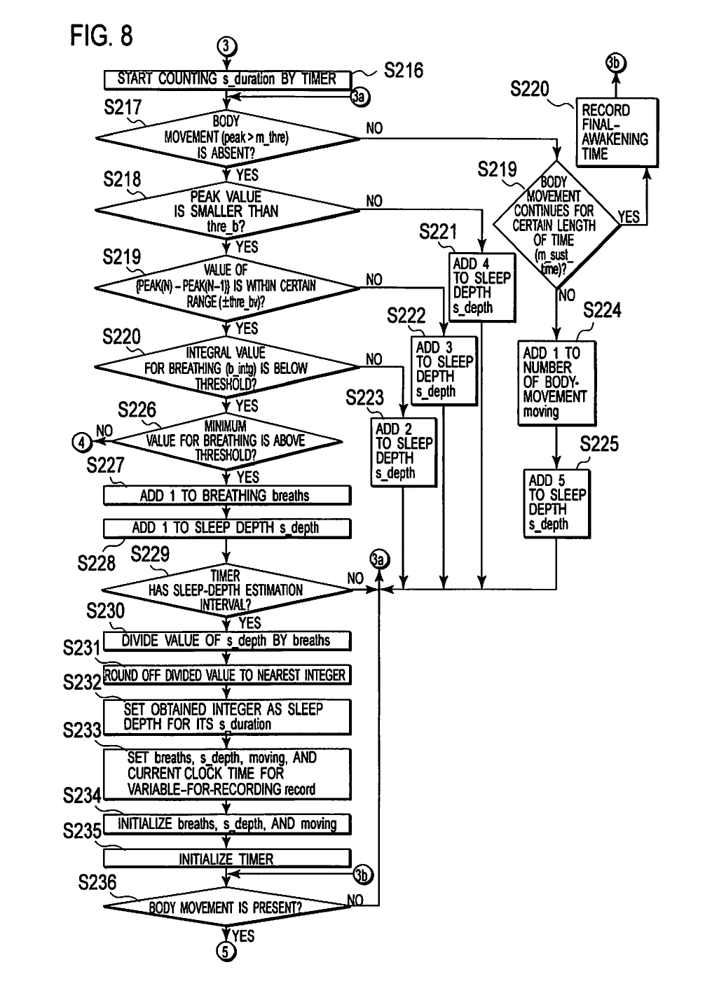

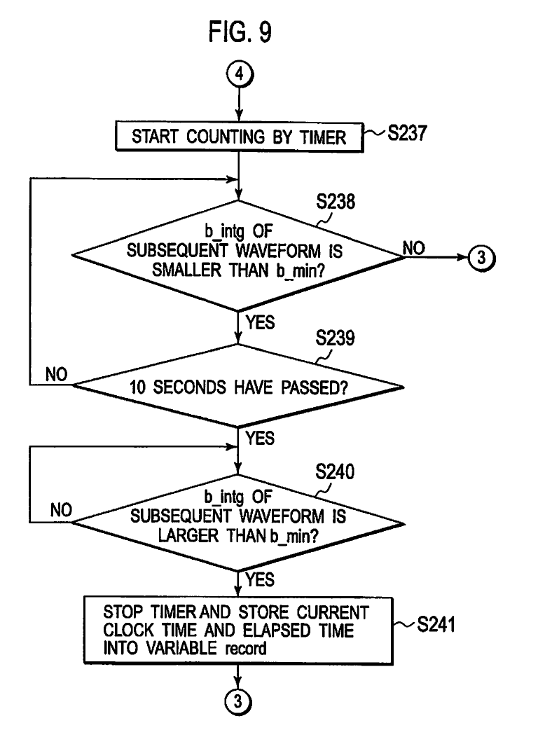

Sensor-data converter 111 judges the sleep depth of the user through the processing at steps S216 to S228 shown in FIG. 8. As the body movement is smaller at step S217, the sleep depth to be set becomes larger. Subsequently, as the breathing quantity is larger, the sleep depth to be set becomes larger. Note that the integral value for breathing mentioned at step S220 is defined as the area of the respiratory waveform as FIG. 12 shows. In addition, when the minimum value for breathing mentioned at step S226 is larger than the threshold value, what comes next is an SAS processing shown in FIG. 9.

The processing for judging the user's sleep depth will be described below with reference to the flowchart shown in FIG. 8. Firstly, at step S216, counting the period (s_duration) by the timer is started. Next, it is judged whether or not the peak value of the amplitude representing the body movement (peak) is larger than the threshold value (m_thre) (at step S217). When it is judged that there is a peak value of the amplitude representing the body movement (peak) which is larger than the threshold value (m_thre), it is then judged whether or not the body movement continues for a certain length of time (m_sust_time) (at step S219). When it is judged that such body movement continues for the certain length of time, it is judged that the user becomes awakened, and the final-awakening time is obtained and recorded (at step S220). The data on the final-awakening time is stored in parameter accumulator 113. When it is judged, at step S219, that such body movement does not continue for the certain length of time, the number of body-movement (moving) is incremented by 1 (at step S224), 5 is added to the sleep depth (s_depth), and then the operational flow proceeds back to step S217. Conversely, when it is judged, at step S217, that there is no peak value of the amplitude representing the body movement (peak) which is larger than the threshold value (m₁₃ thre), it is then judged whether or not the peak value (peak) is smaller than a threshold value (thre₁₃ b) (at step S218). When it is judged the peak value (peak) is equal to or larger than the threshold value (thre_b), 4 is added to the sleep depth (s_depth) (at step S221), and the operational flow proceeds back to step S217. Conversely, when the peak value (peak) is smaller than the threshold value (thre_b), it is then judged whether or not the variation of the peak value of the body-movement waveform (peak (n)-peak (n-1)) is within a predetermined range (there_bv) (at step S219). When it is judged that the variation is out of the predetermined range (there_bv), 3 is added to the sleep depth (s_depth) (at step S222), and the operational flow proceeds back to step S217. Conversely, when the variation is within the predetermined range (there_bv), it is then judged whether or not the integral value for breathing (b_intg) is smaller than a predetermined threshold value (at step S220). When it is judged that the integral value for breathing (b_intg) is equal to or larger than the predetermined threshold value, 2 is added to the sleep depth (s_depth), and the operational flow proceeds back to step S217. Conversely, when the integral value for breathing (b_intg) is smaller than the predetermined threshold value, it is then judged whether or not the minimum value for breathing (b_min) is larger than a predetermined threshold value. When the minimum value for breathing (b_min) is not larger than the predetermined threshold value, the operational flow proceeds to step S237, which will be described later. Conversely, when the minimum value for breathing (b_min) is larger than the predetermined threshold value, 1 is added to the breathing (breaths) (at step S227), and 1 is added to the sleep depth (s_depth) (at step S228).

From step S229 to step S236 of FIG. 8, sensor-data converter 111 performs the processing of recording the sleep depth. The processing of recording the sleep depth will be described below with reference to the flowchart of FIG. 8. Initially a determination is made for an estimation sleep time interval. When the timer shows an interval smaller than the predetermined interval, the operational flow proceeds back to step S217. Conversely, when it is judged the timer has the predetermined interval, the value of the sleep depth (s_depth) is then divided by the breathing (breaths) (at step S230), the resultant value is rounded off to the nearest integer (at step S231), and then the integer thus obtained is used as the sleep depth for the period (s_duration) (at step S232). Subsequently, the data on the breathing (breaths), on the sleep depth (s_depth), on the number of the body-movement (moving), and on the current time are stored in the parameters for recording (at step S233). After storage, these parameters are initialized (at step S234), and the timer is initialized (at step S235). It is then judged whether or not there is any body movement (at step S236). When there is no body movement, the operational flow proceeds back to step S217. Conversely, when there is the body movement, the operational flow proceeds to step S242, which will be described later.

From step S237 to step S241 of FIG. 9, sensor-data converter 111 judges the apneic period and records the apneic period. The processing of judging the apneic period will be described below with reference to the flowchart of FIG. 9. Firstly, counting is started by the timer (at step S237). Then, it is judged whether or not the integral value of breathing (b_intg) of the subsequent waveform is smaller than a predetermined threshold, which is in this case the minimum value of breathing (b_min) (at step S238). When the integral value of the breathing (b_intg) is larger than the minimum value of the breathing (b_min), the operational flow proceeds back to step S216. Conversely, when the integral value of the breathing (b_intg) is equal to or smaller than the minimum value of the breathing (b_min), it is then judged whether or not a predetermined length of time has passed (at step S239). In this embodiment, for example, it is judged whether or not 10 seconds have passed. When it is judged that the predetermined length of time has not passed, the operational flow proceeds back again to step S238. Conversely, when it is judged that the predetermined length of time has passed, it is then judged whether the integral value of the breathing (b_intg) of the subsequent waveform is larger than the minimum value of the breathing (b_min) (at step S240). When it is detected, at this step S240, that the integral value of the breathing (b_intg) is larger than the minimum value of the breathing (b_min), the timer that has been started at step S237 is stopped at that moment, and the current clock time and the elapsed time are stored in the parameter record (at step S241). Once the above-described series of processing is finished, the operational flow proceeds back to step S216.

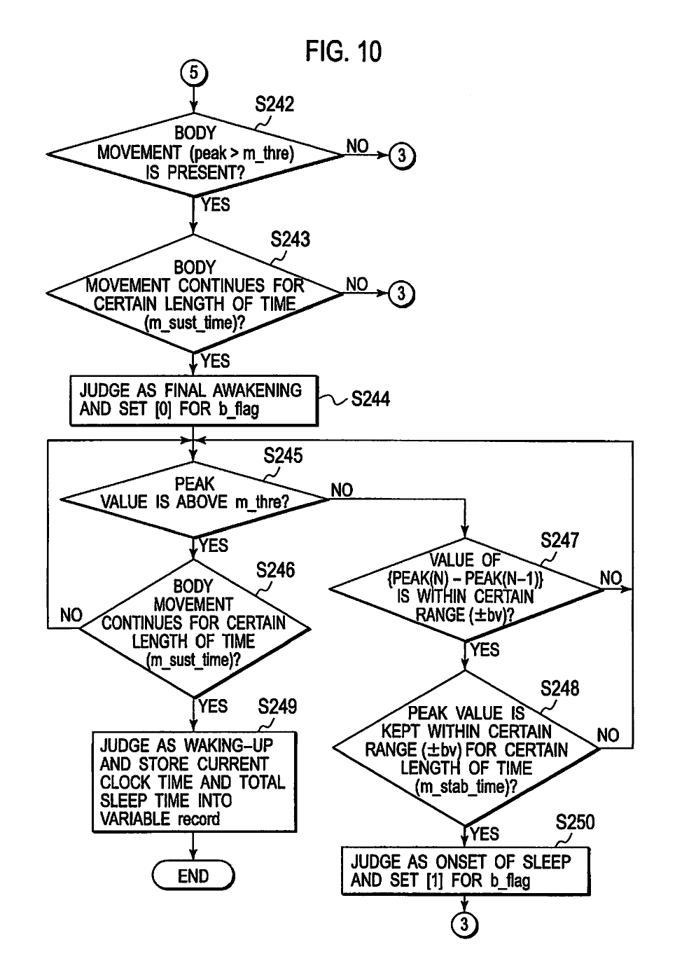

From step S242 to step S250 of FIG. 10, sensor-data converter 111 judges which of the following states the user is in: the state where the user is awakened finally; or the state where the user wakes up, and records the wake-up time or total sleep time. The processing of judging whether the user is in the finally-awakened state, or the waking-up state will be described below with reference to the flowchart of FIG. 10. Firstly, it is judged whether or not there is a body movement. This judgment is performed by judging whether or not the peak value of the amplitude representing the body movement (peak) is larger than the threshold value (m_thre). When the judgment result shows that the peak value (peak) is smaller than the threshold value (m_thre), the operational flow proceeds back to step S217. Conversely, when the peak value (peak) is equal to or larger than the threshold value (m_thre), it is then judged whether or not the body movement continues for the certain length of time (m_sust_time) (at step S243). When it is judged that the body movement does not continue for the certain length of time, the operational flow proceeds back to step S217. Conversely, when it is judged that the body movement continues for the certain length of time, it is judged that the user has awakened, and the flag (b_flag) is set at 0 (at step S244). Next, it is judged whether the peak value of the amplitude representing the body movement (peak) is larger than the threshold value (m_thre) (at step S245). When the peak value is equal to or smaller than the threshold value, it is then judged whether or not the variation of peak values of the body-movement waveform (peak (n)-peak (n-1)) is within the predetermined range (bv) (at step S247). When it is judged that the variation (peak (n)-peak (n-1)) is out of the predetermined range (bv), the operational flow proceeds back to step S245. Conversely, when it is judged that the variation (peak (n)-peak (n-1)) is within the predetermined range (bv), it is then judged whether or not a state where the peak values (peak) of the body movement within the predetermined range (bv) continues for the certain length of time (b_stab_time) (at step S248). When it is judged that such a state does not continue for the certain length of time (b_stab_time), the operational flow proceeds back to step S245. Conversely, when it is judged that such body movement continues for the certain length of time (b_stab_time), it is judged that the user starts sleeping. Then, the flag (b_flag) is set at 1 (at step S250), and the operational flow proceeds back to step S216. On the other hand, it is determined, at step S245, that the peak value (peak) is larger than the threshold value (m_thre), it is then judged whether or not the body movement continues for the certain length of time (m_sust_time) (at step S246). When it is judged that the body movement does not continue for the certain period of time, the operational flow proceeds back to step S245. Conversely, when it is judged that the body movement continues for the certain period of time, it is judged that the user wakes up, and the current clock time and the total sleep time are stored in the parameters record (at step S249).

### (3.3) Flow of Trend Prediction Processing

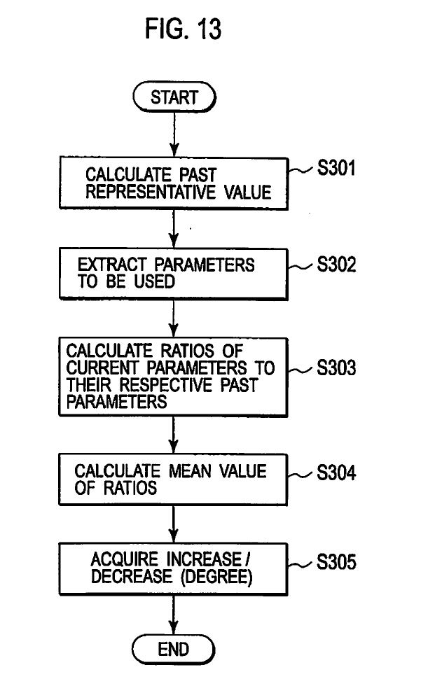

FIG. 13 is a flowchart illustrating a procedure of trend prediction processing performed by trend prediction device 100A.

At step S301, reference-parameter calculator 114 calculates both representative values of past sleep-related parameters P1 and representative values of past lifestyle-related parameters P2, which are accumulated in parameter accumulator 113, as reference parameters P3. Note that mean values are used as the representative values in this embodiment.

At step S302, parameter extractor 116 refers to parameter-to-be-used table T1, and extracts some of sleep-related parameters P1 and of lifestyle-related parameters P3 that correspond to the kind of physical data to be predicted. In addition, parameter extractor 116 extracts reference parameters P3 for the extracted ones of sleep-related parameters P1 and of lifestyle-related parameters P2.

At step S303, parameter comparator 117 calculates the ratios R of the sleep-related parameters P1 and the lifestyle-related parameters P2 extracted by parameter extractor 116 to their respective reference parameters P3.

At step S304, mean-value calculator 118 calculates the mean value A of the ratios R calculated by parameter comparator 117.

At step S305, prediction unit 120 refers to table for judgment T2, and acquires information on the trend and its degree corresponding to the mean value A calculated by mean-value calculator 118. The information thus acquired is passed on to display unit 121.

### (4) Effects

According to this embodiment, the judgment of physical-data trend is performed using not only sleep-related parameters P1 but also lifestyle-related parameters P2. For this reason, variation trend for the user's physical data can be predicted by taking account of both the state where the user is in sleep and the state where the user is awake. As a consequence, the variation trend for the physical data can be predicted with higher accuracy.

In addition, whether the physical data has an increased trend or a decreased trend is judged on the basis of the results of both the comparison of sleep-related parameters P1 with reference parameters P3 and the comparison of lifestyle-related parameters P2 with reference parameters P3. Further, no information on bed B is necessary for the judgment. For these reasons, the trend prediction device 100A of this embodiment is highly versatile.

According to this embodiment, display unit 121 displays the information on whether the physical data has an increased trend or a decreased trend. For this reason, the users can promote their health and prevent disease more actively.

According to this embodiment, not only an increase or decrease in the physical data trend but also the degree of the change in the physical-data trend can be judged. For this reason, the variation trend for the physical data can be predicted with even higher accuracy.

According to this embodiment, reference parameters P3 are calculated both from past sleep-related parameters P1 and from past lifestyle-related parameters P2. For this reason, reference parameters P3 can be adapted to the individual user. As a consequence, the trend prediction device 100A can be more versatile. In addition, the use of reference parameters P3 that are adapted to the individual user improves the accuracy of the prediction for the variation trend of the physical data.

According to this embodiment, different parameters of sleep-related parameters P1 and of lifestyle-related parameters P2 are extracted depending on which trend of the physical data of the user is to be predicted among the blood pressure, the blood-sugar level, the body weight, or the body-fat percentage. For this reason, the parameters to be used can be selected so that the selected ones can be appropriate for the blood pressure, the blood-sugar level, the body weight, and the body-fat percentage, respectively. As a consequence, the variation trend for the physical data can be predicted with higher accuracy.

As has been described thus far, according to this embodiment, it is possible to provide the trend prediction device 100A that is highly versatile, and is capable of improving the accuracy of predicting the variation trend of the physical state of the user.

### Second Embodiment

The description in a second embodiment focuses mainly on those different from the first embodiment described above. No description that has already been given in the first embodiment will be repeated. In the second embodiment, descriptions will be given below in the following order: (1) the functional block configuration of a trend prediction device; (2) the processing of factor analysis; and (3) effects.

### (1) Functional Block Configuration of Trend Prediction Device

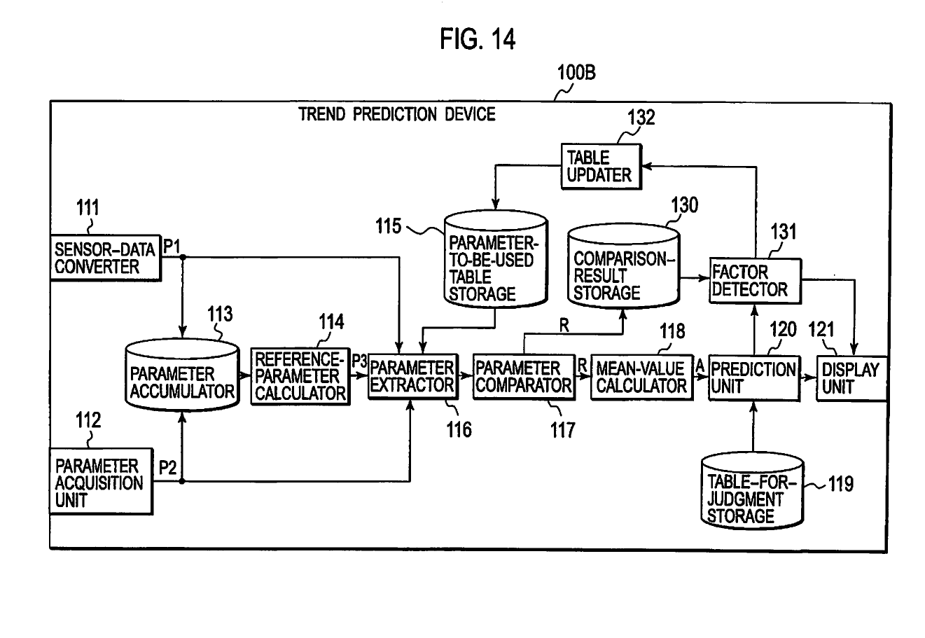

FIG. 14 is a diagram illustrating the functional-block configuration of trend prediction device 100B according to the second embodiment. Trend prediction device 100B differs from trend prediction device 100A in the first embodiment described above in that trend prediction device 100B includes comparison-result storage 130, factor detector 131, and table updater 132.

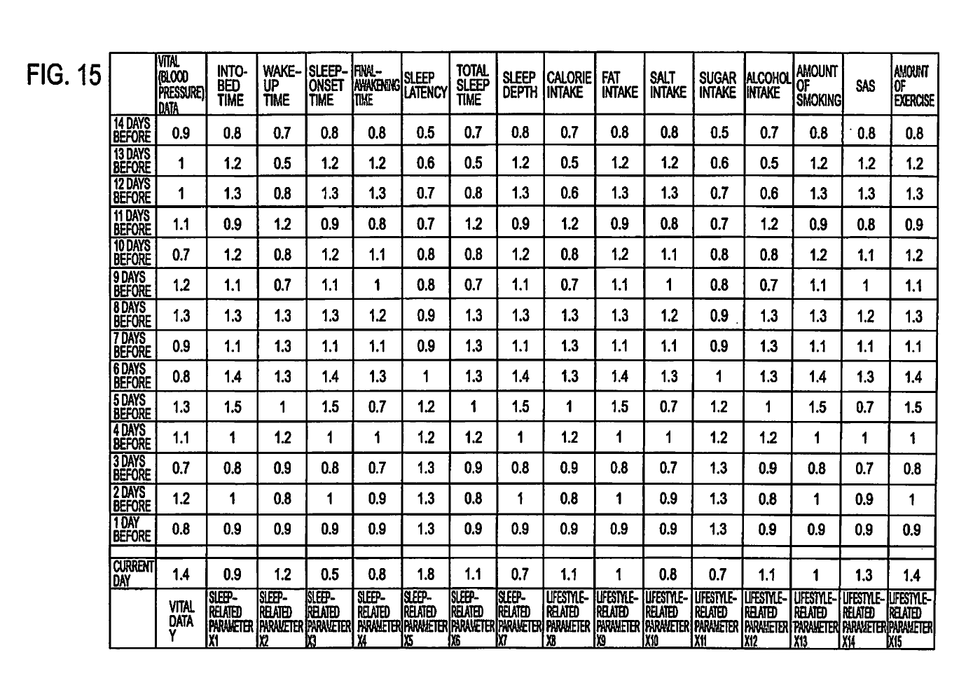

Comparison-result storage 130 is configured to store the past ratios R calculated by parameter comparator 117 as FIG. 15 shows. The ratios R thus stored correspond to all of sleep-related parameters P1 and lifestyle-related parameters P2. In the example shown in FIG. 15, the ratios R for the last 14 days are stored.

Factor detector 131 is configured to detect, among all of sleep-related parameters P1 and lifestyle-related parameters P2, factor parameters P4 that are the factors causing the change in the physical-data trend on the basis of the results of the prediction performed by prediction unit 120. The detection of factor parameters P4 can be accomplished by detecting the parameters that are highly relevant to the change in the physical-data trend by means of the known multiple-regression analysis method. In FIG. 15, when Y represents the trend of physical data and X₁, X₂, . . . represent parameters, the following formula (1) holds true.

Y=X₁×A₁+X₂×A₂+X₃×A₃+ . . . +X₁₅×A₁₅+B (1)

In the formula (1), A₁, A₂, . . . are weighting coefficients that are uniquely given to the respective parameters, and B is the correction value.

Display unit 121 is configured to display the information on factor parameters P4 detected by factor detector 131. To put it differently, display unit 121 in this embodiment functions as factor notification unit that is configured to notify the user of factor parameters P4 detected by factor detector 131.

Table updater 132 is configured to update parameter-to-be-used table T1 by use of factor parameters P4 detected by factor detector 131. When a high relevancy is found between the variation of a physical data and the variation of a parameter that is not included in parameter-to-be-used table T1, the parameter is added to parameter-to-be-used table T1. In addition, table updater 132 is capable of deleting any parameter included in parameter-to-be-used table T1 when the parameter shows low relevancy to the variation of the physical data.

### (2) Flow of Factor-Analysis Processing

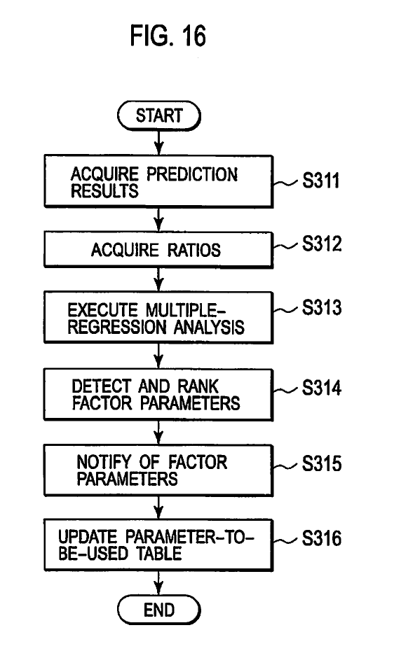

FIG. 16 is a flowchart illustrating the procedure of factor analysis processing performed by trend prediction device 100B.

At step S311, factor detector 131 acquires, from prediction unit 120, information indicating the trend and its degree of the physical data.

At step S312, factor detector 131 acquires the ratios R of the current day to the past stored in comparative-result storage 130.

At step S313, factor detector 131 executes a multiple-regression analysis in accordance with the formula (1) on the basis of the trend and its degree of the physical data as well as of the ratios R of the current day to the past.

At step S314, factor detector 131 detects factor parameters P4, and ranks detected factor parameters P4 in the order of their relevancy from high to low.

At step S315, display unit 121 displays factor parameters P4 detected by factor detector 131 in the order of relevancy from high to low.

At step S316, table updater 132 updates parameter-to-be-used table T1 by use of factor parameters P4 detected by factor detector 131.

### (3) Effects

According to the second embodiment, parameter-to-be-used table T1 is updated by use of detection factor parameters P4 that are the factors causing the change in the trend of the physical data. For this reason, parameter-to-be-used table T1 can be optimized. As a consequence, trend prediction device 100B can be more versatile, and the trend for the physical data can be predicted with higher accuracy.

According to the second embodiment, the user is notified of factor parameters P4. For this reason, the users can promote their health and prevent disease more actively.

### Other Embodiments

As described above, the first and the second embodiments have been described. While the description and the drawings given above form parts of the disclosure, neither the description nor the drawings can limit the invention. Those skilled in the art can arrive at various alternative embodiments, examples, and application techniques.

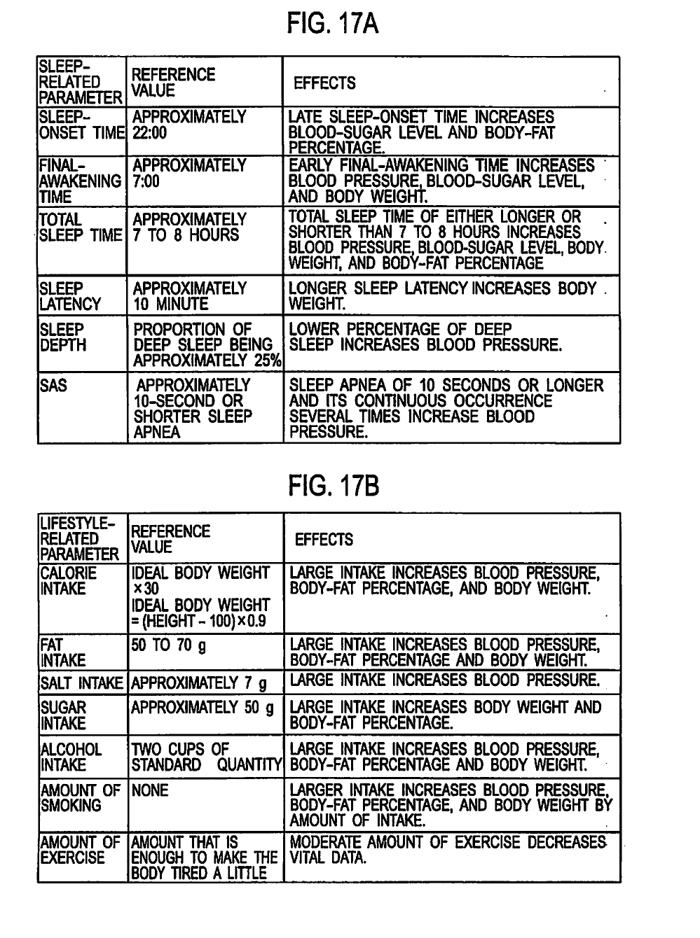

In the above-described embodiments, reference parameters P3 are created from past sleep-related parameters P1 and past lifestyle-related parameters P2. As shown in an alternative configuration in FIGS. 17A and 17B, reference parameters P3 may be given in advance and no change of reference parameters P3 is permitted. FIG. 17A shows reference parameters P3 for sleep-related parameters P1 while FIG. 17B shows reference parameters P3 for lifestyle-related parameters P2.

In the examples of the first and the second embodiments, parameter acquisition unit 112 acquires lifestyle-related parameters P2 by means of the inputs by the user. Instead, lifestyle-related parameters P2 may be acquired by means of the information detected by any other sensors than sleep sensor 200.

In addition, in the above-described embodiments, sleep-related parameters P1 are obtained by converting the sensor data. In an alternative configuration, sleep-related parameters P1 can be obtained by means of the inputs by the user.

In the description of the second embodiment, the method of updating the table is on the basis of the result of prediction performed by prediction unit 120. Instead, parameter-to-be-used table T1 may be updated on the basis not only of the result of prediction performed by prediction unit 120 but also of the information on the trend obtained from the measured values of the physical data.

In the above-described embodiments, sleep sensor 200 is directly connected to trend prediction device 100A or 100B. Instead, a network may be employed to connect sleep sensor 200 to trend prediction device 100A or 100B.

The invention includes other embodiments in addition to the above-described embodiments without departing from the spirit of the invention. The embodiments are to be considered in all respects as illustrative, and not restrictive. The scope of the invention is indicated by the appended claims rather than by the foregoing description. Hence, all configurations including the meaning and range within equivalent arrangements of the claims are intended to be embraced in the invention.

What is claimed is:

1. A trend prediction device that indicates the physical conditions of a user, comprising:
a first parameter acquisition unit configured to acquire values of a first judgment parameter for the user's sleep, which is a quantified factor affecting the physical data;
a second parameter acquisition unit configured to acquire values of a second judgment parameter for an action of the user during non-sleeping, which is a quantified factor affecting the physical data;
a parameter accumulator configured to accumulate the acquired values of the first and second judgment parameters;
a reference parameter calculator configured to calculate a first reference parameter from the values of the first judgment parameter accumulated in the parameter accumulator and to calculate a second reference parameter on the basis of the values of the second judgment parameter accumulated in the parameter accumulator;
a parameter comparator configured to compare each of the values of the first judgment parameter with the first reference parameter and to compare each of the values of the second judgment parameter with the second reference parameter; and
a trend judgment unit configured to detect an increased or decreased trend in a parameter by comparing each value of the first judgment parameter with the first reference parameter and from comparing each value of the second judgment parameter with the second reference parameter.

2. The device of claim 1, wherein the first judgment parameter is obtained by converting sensor data detected by a sleep sensor disposed in a bed.

3. The device of claim 1, wherein the parameter comparator calculates a set of ratios of the first reference parameter with the first judgment parameter values and a set of ratios of the second reference parameter with the second judgment parameter values.

4. The device of claim 3, wherein
the trend judgment unit comprises:
a mean-value calculator configured to calculate a mean value of each set of ratios calculated by the parameter comparator; and
a prediction unit configured to determine a degree of increased or decreased trend of physical data from the mean values calculated by the mean-value calculator.

5. The device of claim 4, wherein
the trend judgment unit further comprises storage for a judgment-table configured to store the mean values of ratios between the values of the first reference parameter and the first judgment parameter, and
the prediction unit judges the degree of increase or decrease in physical data from the mean values calculated by the mean-value calculator and from values stored in the judgment table.

6. The device of claim 1, further comprising:
a parameter extractor configured to extract, at least one first judgment parameter value obtained by the first parameter acquisition unit and at least one second judgment parameter value obtained by the second parameter acquisition unit, wherein
the parameter comparator compares the first judgment parameter extracted by the parameter extractor with the first reference parameter and compares the second judgment parameter extracted by the parameter extractor with the second reference parameter.

7. The device of claim 6, further comprising:
a table holding unit configured to hold a parameter-to-be-used table that associates physical data to be predicted with the first judgment parameter and the second judgment parameter that are to be extracted by the parameter extractor;
a factor detector configured to detect, among the first judgment parameters and the second judgment parameters, a factor parameter that causes a change in the trend of the physical data, from a judgment by the trend judgment unit; and
a table updater configured to update the parameter-to-be-used table by use of the factor parameter detected by the factor detector.

8. The device of claim 1, further comprising a judgment-result notification unit configured to notify the user of the result of a judgment performed by the trend judgment unit.

9. The device of claim 1, wherein the reference-parameter calculator calculates the first reference parameter from any one of a mean value, a median value, and a mode value of the past values of the first judgment parameter accumulated in the parameter accumulator.

10. A trend prediction system that indicates a physical condition of a user, comprising:
a sleep sensor installed in abed, configured to detect vibration from a user and to output sensor data;
a first parameter acquisition unit configured to acquire the sensor data as a first judgment parameter from the sleep sensor.
a second parameter acquisition unit configured to acquire values of a second judgment parameter indicating an action of the user during non-sleeping, which is a quantified factor affecting the physical data;
a parameter accumulator configured to accumulate the acquired values of the first judgment parameter and the acquired values of the second judgment parameter;
a reference parameter calculator configured to calculate a first reference parameter from the values of the first judgment parameter accumulated in the parameter accumulator and to calculate a second reference parameter from the values of the second judgment parameter accumulated in the parameter accumulator;
a parameter comparator configured to compare each value of the first judgment parameter with the first reference parameter and to compare value of the second judgment parameter with the second reference parameter; and
a trend judgment unit configured to detect an increased or decreased trend from comparing each value of the first judgment parameter with the first reference parameter from comparing each value of the second judgment parameter with the second reference parameter.

11. The system of claim 10, wherein the parameter comparator calculates a set of ratios of the first reference parameter with the first judgment parameter as well as a set of ratios of the second reference parameter with the second judgment parameter.

12. The system of claim 11, wherein
the trend judgment unit comprises:
a mean-value calculator configured to calculate a mean value of each set of ratios calculated by the parameter comparator; and
a prediction unit configured to detect an increased or decreased trend of physical data from the mean value calculated by the mean-value calculator.

13. The system of claim 12, wherein
the trend judgment unit further comprises a judgment-table storage configured to store the mean value of the ratios between the values of the first reference parameter and the first judgment parameter, and wherein
the prediction unit detects increased or decreased trend of physical data from the mean value calculated by the mean-value calculator and from the degree stored in the judgment table.

14. The system of claim 10, further comprising:
a parameter extractor configured to extract, depending on a type of the physical data to be predicted, at least a value of the first judgment parameters obtained by the first parameter acquisition unit and at least a value of the second judgment parameters obtained by the second parameter acquisition unit, wherein
the parameter comparator compares the first judgment parameter extracted by the parameter extractor with the first reference parameter and compares the second judgment parameter extracted by the parameter extractor with the second reference parameter.

15. The system of claim 14, further comprising:
a table holding unit configured to hold a parameter-to-be-used table that associates the kind of the physical data to be predicted with the first judgment parameter and the second judgment parameter that are to be extracted by the parameter extractor;
a factor detector configured to detect, among the first judgment parameters and the of second judgment parameters, a factor parameter that causes a change in the physical data, from the judgment performed by the trend judgment unit; and
a table updater configured to update the parameter-to-be-used table by use of the factor parameter detected by the factor detector.

16. The system of claim 10, further comprising a judgment-result notification unit configured to notify the user of the judgment performed by the trend judgment unit.

17. The system of claim 10, wherein the reference-parameter calculator calculates the first reference parameter from any one of a mean value, a median value, and a mode value of the past values of the first judgment parameter accumulated in the parameter accumulator.
